(19) United States
(12) Patent Application Publication
(10) Pub. No.: US 2020/0008914 A1
(43) Pub. Date: Jan. 9, 2020

(54) A DENTALY LIGHT POLYMERIZATION DEVICE

(71) Applicant: 3M INNOVATIVE PROPERTIES COMPANY, St. Paul, MN (US)

(72) Inventors: Rudolf Schmid, Eichenau (DE); Korbinian Gerlach, Gauting (DE); Stefan K. Welker, Geltendorf (DE)

(21) Appl. No.: 16/493,890

(22) PCT Filed: Mar. 15, 2018
(86) PCT No.: PCT/US2018/022539

§ 371 Date: Sep. 13, 2019

(30) Foreign Application Priority Data
Mar. 17, 2017 (EP) 17161495.1

(51) Int. Cl. A61C 13/15; A61B 1/24; A61B 1/00; A61B 1/06

(52) U.S. Cl. CPC A61C 19/004; A61B 1/0684; A61B 1/00096; A61B 1/24

(57) ABSTRACT

A dental light polymerization device has an intra-oral tip portion and a handle portion. A polymerization light source and a camera are accommodated in the dental light polymerization device. The camera has an image input arranged within the tip portion and the polymerization light source is formed by a plurality of polymerization light outputs that are arranged around the image input. The dental light polymerization device further has a light refraction panel in which a plurality of lenses are interspersed. The light refraction panel is arranged with the lenses being each positioned in front of a corresponding polymerization light output of the plurality of polymerization light outputs.

### FIELD OF THE INVENTION

The invention relates to a dental light polymerization device which has a polymerization light source and a camera. A light refraction panel that has a plurality of integrated lenses is arranged in front of polymerization light outputs that form the polymerization light source.

### BACKGROUND ART

Light hardenable or light curable materials are widely used in dentistry for the restoration of teeth. Many of such materials are made to provide optical instructions that resemble those of natural teeth. Further such materials typically can be placed precisely and conveniently, can be hardened instantly, and the hardened material is typically relatively durable. Accordingly these materials are favored alternatives to less pleasant looking and over time self-hardening materials, like for example amalgam.

Light hardenable materials often include a polymerizable matrix material and filler materials including colorants, and may initially be generally soft or flowable so that they can be applied in a desired location and shape. For example, for restoration of a tooth the dental material may be filled into a tooth cavity and shaped so that the restored tooth resembles a natural tooth. Once the desired shape has been formed, the material may be cured by exposing it to light of a desired wavelength. The light typically activates photoinitiators in the dental material that cause the matrix material to polymerize.

The use of dental materials that are hardenable by blue light of a wavelength of between about 450 and 500 nm (nanometers) has become common in dentistry. Accordingly, light-emitting devices used for hardening such dental materials typically emit light at such wavelengths. Such a light-emitting device is for example available from 3M Deutschland GmbH, Germany, under the trade designation Elipar™ S10.

Further, it has been proposed provide a dental light device with a camera. For example, WO 2014/043488 discloses a dental irradiation device having a first light emitting unit for emitting blue light for light hardening of a dental material and second light emitting unit. The device further has an image sensing unit. The device can be used for simultaneous illumination and image capturing.

Although the addition of a camera provides useful additional functions there is a desire for a dental light device in which the light polymerization function is not affected but rather maximized.

### SUMMARY OF THE INVENTION

The invention relates to a dental light polymerization device that comprises an intra-oral tip portion (further referred to as “tip portion” herein) and a handle portion. The dental light polymerization device comprises a polymerization light source and a camera. The camera has an image input that is arranged within the tip portion. Further, the polymerization light source comprises a plurality of polymerization light outputs that are arranged around the image input. The dental light polymerization device further comprises a light refraction panel in which a plurality of lenses are interspersed. The light refraction panel is arranged with the lenses being each positioned in front of a corresponding polymerization light output of the plurality of polymerization light outputs.

The invention is advantageous in that it provides a dental light polymerization device that is capable of emitting generally uniform polymerization light. Such uniform polymerization light helps initiating polymerization at a generally uniform level in any portion of light hardenable material that is exposed to the polymerization light. In particular, peaks or voids in the polymerization light beam can be minimized. Further, although the dental light polymerization device has more than a single light output, the light refraction panel enables a conversion of the light emitted by the multiple light outputs into a generally uniform polymerization light beam. Furthermore, the light refraction panel helps facilitating the assembly and positioning of several lenses relative to multiple light outputs.

The term “the light refraction panel is arranged with the lenses being each positioned in front of a corresponding polymerization light output” means that the lenses and the polymerization light outputs are arranged relative to each other such hat light emitted from the polymerization light outputs passes through the lenses. Preferably, the light refraction panel is spaced from the polymerization light outputs. The light refraction panel may be spaced from the polymerization light outputs by a distance of between 1 mm and 4 mm, preferably 2 mm. Thus, any transfer of heat as eventually generated by the polymerization light outputs toward the light refraction panel is minimized.

The polymerization light outputs are preferably arranged circularly around the image input. Further, the polymerization light outputs may be uniformly distributed circularly around the image input. For example, the dental light polymerization device may have four polymerization light outputs that are circularly arranged at 0 degrees, 90 degrees, 180 degrees, 270 degrees around the image input. The diameter of the circle of the circular arrangement is preferably about 10 mm. Further, the image input is preferably arranged in the middle of the circle of the circular arrangement.

In an embodiment the polymerization light outputs are arranged within the tip portion. Preferably, the tip portion has a free end in which the polymerization light outputs are arranged.

In an embodiment the lenses are freeform lenses. The freeform lenses are preferably customized with respect to both, the optical characteristics of the light emitted from the light outputs and the desired properties of the light beam emitted from the lenses in combination. According to the present invention the polymerization light emitted from the dental light polymerization device is preferably parallel and uniformly distributed. Based on these desired characteristics the lenses are designed for, in combination, converting any non-parallel and non-uniform light emitted from the individual polymerization light outputs into one common parallel and uniform light beam. Accordingly the lenses typically have a lens surface topography that is customized. In particular the surfaces of the lenses preferably do not have one common axis of rotation. In other words the freeform lenses are preferably not rotation-symmetric. Each lens is preferably based on the same freeform or may have the same freeform. Thus, in combination the lenses preferably provide for a parallel light beam having a uniform light intensity with respect to a cross-sectional area perpendicular to an optical axis along which the light beam is emitted.

In a further embodiment each of the lenses have a first optical characteristic. Further, the light refraction panel preferably comprises a plurality of intermediate portions that are arranged between the lenses and which have a second optical characteristic. Preferably the second optical characteristic differs from the first optical characteristic. The first optical characteristic preferably comprises or forms a focusing characteristic that converts a non-parallel (in particular diverging) light beam from the respective polymerization light output into a parallel light beam or a substantially parallel light beam.

In a further embodiment the dental light polymerization device further comprises an illumination light source. The illumination light source comprises a plurality of illumination light outputs that are arranged around the image input. The illumination light outputs are preferably arranged within the tip portion, preferably within the free end of the tip portion. Preferably, the polymerization light outputs and the illumination light outputs are alternately arranged around the image input. Preferably, the light refraction panel is arranged with the intermediate portions each positioned in front of a corresponding illumination light output of the plurality of illumination light outputs.

The illumination light outputs are preferably arranged circularly around the image input. Further, the illumination light outputs may be uniformly distributed circularly around the image input. For example, the dental light polymerization device may have four illumination light outputs that are circularly arranged at 45 degrees, 135 degrees, 225 degrees, 315 degrees around the image input. The circle of the circular arrangement preferably corresponds in size and arrangement to the circle of the circular arrangement of the polymerization light outputs.

In an embodiment the intermediate portions and the lenses are monolithically formed. In particular the light refraction panel preferably forms one monolithic piece, at least part of which is formed by the lenses and the intermediate portions. The light refraction panel may be made of polymethyl methacrylate (PMMA), polycarbonate (PC) or cycloolefin-copolymer (COC), for example.

In a further embodiment, the lenses are oriented such that the light beams exiting the light refraction panel are generally parallel. Hence, an overall polymerization light beam emitted from the dental light polymerization device is preferably formed of generally parallel light.

In a further embodiment the intermediate portions are part of a light transmissive plane-parallel portion of the light refraction panel.

In one embodiment the second optical characteristic comprises a light dispersion characteristic. For example, the intermediate portions may be made of transparent material, but at least one surface of the intermediate portions may have a roughened surface. Such a roughened surface may have a sandblasted structure, for example. Accordingly, the intermediate portions are overall not transparent but only translucent. Thus, the intermediate portions provide for the light that passes through the intermediate portions to disperse.

In one embodiment each polymerization light output is provided by a blue LED. Further, each illumination light output is preferably provided by a white LED. The blue and white LEDs as well as the camera are preferably arranged within the tip portion.

A “blue LED” as referred to herein is a LED which emits blue light when activated. Further, a “white LED” as referred to herein is a LED which emits white light when activated.

For the purpose of the present specification the term “blue light” refers to light having a wavelength within the range of about 430 nm to 500 nm, preferably within a range of about 430 nm to 480 nm. Blue light preferably predominantly consists of light within a range of about 430 nm to 480 nm. The blue light may particularly not comprise light having a wavelength outside the range of about 430 nm to 480 nm at a substantial intensity or at all. In particular, blue light may have a first portion of light within a range of about 430 nm to 480 nm and preferably does not have a significant second light portion within a range of 570 nm and 590 nm, wherein the maximum intensity of the second portion of light is preferably less than 10% and more preferably less than 1% of the maximum intensity of the first portion of light. Further blue light may not have a significant third light portion within the spectrum of visible light outside the range of 430 nm and 480 nm and outside the range of 570 nm to 590 nm, wherein the maximum intensity of any third portion of light is preferably less than 25% and more preferably less than 20% of the maximum intensity of the first portion of light.

For the purpose of the present specification the term “white light” refers to light having a wavelength within a range of about 380 nm to 780 nm. Although white light may also comprise light at wavelengths overlapping with the range of wavelengths of blue light, the white light preferably does not predominantly consist of light within that range but has significant portions of visible light at wavelengths outside that range.

Accordingly, the polymerization light source is preferably configured for emitting blue light, whereas the illumination light source is preferably configured for emitting white light.

In an embodiment the dental light polymerization device further comprises a first printed circuit board that carries the white and blue LEDs and the camera. The first printed circuit board is preferably arranged within the tip portion. The dental light polymerization device further preferably comprises at least a second printed circuit board that is arranged within the handle portion. The second printed circuit board preferably carries electronic circuitry for electrically operating the LEDs and/or the camera. The first and second printed circuit board are preferably electrically connected via a flexible printed circuit board, but may also be connected by electric wires.

In one embodiment the dental light polymerization device further comprising electronic circuitry that comprises a wireless communication module for at least transmitting images captured by the camera. Thus, images captured by the dental light polymerization device can be displayed substantially real-time on a separate display device (for example a computer screen or tablet).

In one embodiment the light refraction panel has an opening in front of the image input. Accordingly, the image input is preferably not covered by the light refraction panel. Therefore, any light which is dispersed within the light refraction panel (for example due to impurities in the material or due to the material properties itself) does not affect the image capturing via the image input.

### BRIEF DESCRIPTION OF THE FIGURES

FIG. 1 is a perspective view of a dental light polymerization device according to an embodiment of the invention;

FIG. 2 is a perspective cross-sectional view of a detail of a dental light polymerization device according to an embodiment of the invention;

FIG. 3 is a front view onto a tip portion of a dental light polymerization device according to an embodiment of the invention; and

FIG. 4 is a perspective cross-sectional view of a dental light polymerization device according to an embodiment of the invention.

### DETAILED DESCRIPTION OF THE INVENTION

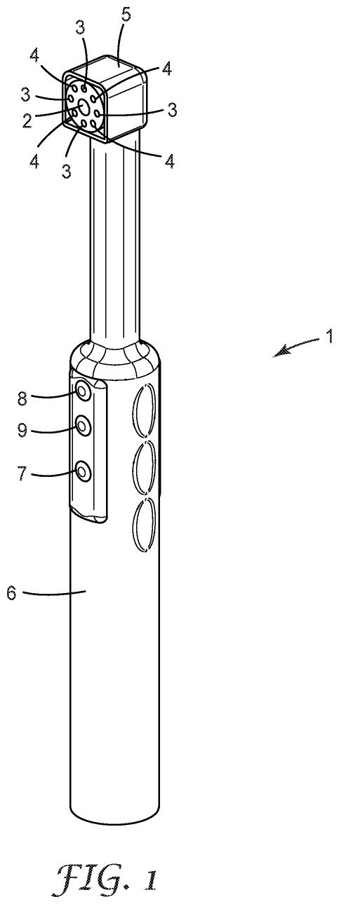

FIG. 1 shows a dental light polymerization device 1. The dental light polymerization device 1 has a tip portion 5 and a handle portion 6. Further, the dental light polymerization device 1 has an image input 2 of a camera, a polymerization light source for emitting blue light and an illumination light source for emitting white light. The polymerization light source comprises a plurality of polymerization light outputs 3. Further, the illumination light source comprises a plurality of illumination light outputs 4. The camera may be a CCD or CMOS based camera, or may be based on any other appropriate technology. Further, the polymerization light source may comprise one or more blue LEDs (Light Emitting Diodes) and the second light source may comprise one or more white LEDs.

In the example, the camera as well as the polymerization and illumination light source are accommodated within the tip portion 5. Other configurations are possible. For example, the camera, the polymerization and/or the illumination light source may be accommodated in the handle portion 6 of the dental light polymerization device 1. In this case one or more light guide(s) may extend between the camera and light sources and the respective image input and/or light outputs.

In particular, in the example the camera forms the image input 2. Further, each of the polymerization light outputs 3 are formed by a respective blue LED, whereas each of the illumination light outputs 4 are formed by a respective white LED.

The light polymerization device 1 in the example has a polymerization light button 8, a timer setting button 7 and a camera button 9. The polymerization light button 8 enables a user to activate the polymerization light source (for example for a duration which can be preset via the timer setting button 7) or to deactivate the polymerization light source. Further, the camera button 9 enables a user to activate or deactivate the camera.

The dental light polymerization device 1 in the example is an overall wireless device, but may in another example be wired. In the example the light polymerization device 1 has a rechargeable battery (not visible). For charging the battery a charging device may be provided (not shown) by which the battery can be charged. For charging the battery the light polymerization device 1 may be connected to the charging device in a contactless manner or by electrical contact-based connection.

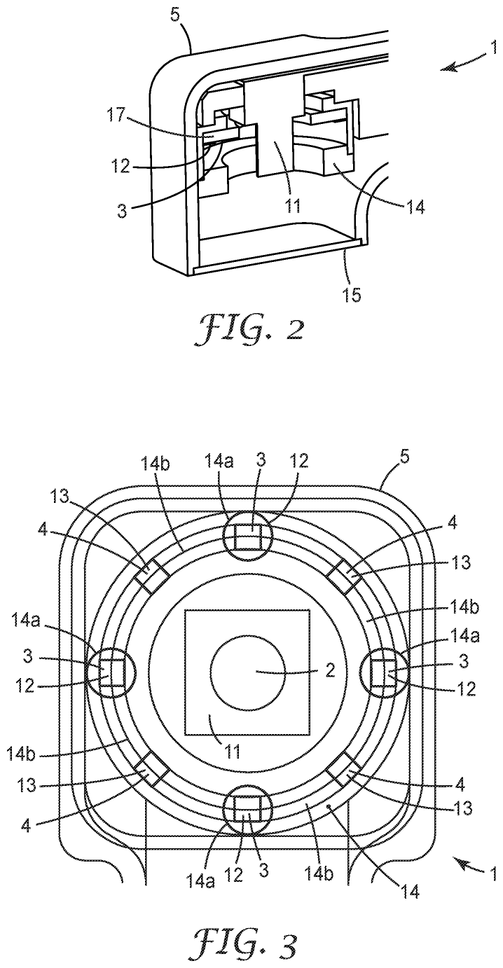

FIG. 2 shows a cross-section of the tip portion 5 of the dental light polymerization device 1 in more detail. The tip portion 5 accommodates the camera 11 which forms the image input 2, the blue LEDs 12 (also designated in FIG. 3) forming the polymerization light outputs 3 and the white LEDs 13 (designated in FIG. 3) forming the illumination light outputs 4. The blue LEDs 12 and the white LEDs 13 are arranged on a common first printed circuit (no. 17 in FIG. 3). The dental light polymerization device 1 further has a light refraction panel 14 for refracting at least the light that is emitted via the polymerization light outputs 4. In the example the light refraction panel 14 is an overall transparent ring shaped structure. The light refraction panel 14 is arranged in front of the polymerization light outputs 4. This means that the light refraction panel 14 is arranged so that the light emitted from the polymerization light outputs 4 passes through the light refraction panel 14. The tip portion 5 in the example is sealed by a transparent plane-parallel cover 15.

FIG. 3 shows the tip portion 5 in a front view. As shown, the polymerization light outputs 3 are arranged around the image input 2. In particular, the polymerization light outputs 3 are uniformly distributed on a circumference around the image input 2. In the example, the dental light polymerization device 1 has four polymerization light outputs 3 which are arranged at 0 degrees, 90 degrees, 180 degrees and 270 degrees on the circumference around the image input 2. Further, the illumination light outputs 4 are also arranged around the image input 2. In particular, the illumination light outputs 4 are uniformly distributed on the same circumference around the image input 2 as the polymerization outputs 3. In the example, the dental light polymerization device 1 has four illumination light outputs 4 which are arranged at 45 degrees, 135 degrees, 225 degrees and 315 degrees on the circumference around the image input 2. In other words the polymerization and illumination light outputs are each distributed at 90 degrees angularly offset from each other and the polymerization light outputs are further angularly offset by 45 degrees from the illumination light outputs. The light refraction panel 14 is interspersed with a plurality of lenses 14a. The distribution of the lenses 14a corresponds to the distribution of the polymerization light outputs 3. Further, preferably the circumferential arrangement of the lenses 14a corresponds to the circumferential arrangement of the polymerization light outputs 3. Thus, each lens 14a covers or is arranged in front of a respective polymerization light output 3.

The light refraction panel 14 comprises a plurality of intermediate portions 14b that are arranged between the lenses 14a. Accordingly, the intermediate portions 14b and the lenses 14a are arranged alternately on the circumference around the image input 2. In the example, the light refraction panel 14 is overall ring-shaped and has two opposite parallel major surfaces, except for surface areas that form part of the lenses 14a. Accordingly, the intermediate portions 14b exhibit opposite parallel surfaces as opposed to the lenses 14a which each have at least one surface that is curved. Therefore, any blue light emitted from the polymerization light outputs 3 and passing through the light refraction panel 14 is converged by the lenses 14a relative to the light entering the light refraction panel 14. Further, any diverging white light emitted from the illumination light outputs 4 and passing through the light refraction panel 14 still diverges when exiting the light refraction panel 14. Thus, an effective light polymerization is enabled while an image can be captured at appropriate illumination of an object from which the image is taken.

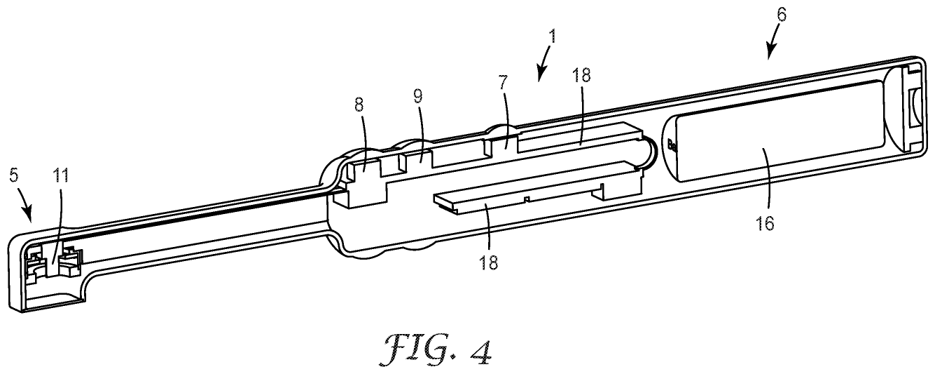

FIG. 4 shows a cross-section of the dental light polymerization device 1. In addition to the camera 11 and the LEDs (not designated in this view) within the tip portion 5, the handle portion 6 accommodated further components of the dental light polymerization device 1. In particular the dental light polymerization device 1 has a (rechargeable) battery 16 and electronic circuitry arranged on a second printed circuit board 18. The second printed circuit board 18 in the example is divided in two portions that are electrically connected with a flexible portion. Thus, the space for accommodation of the second circuit board 18 can be minimized. The electronic circuitry further comprises at least part of the polymerization light button 8, the timer setting button 7 and the camera button 9. The electronic circuitry preferably comprises a wireless communication module for transmitting images captured by the camera to a device outside the dental light irradiation device 1.

1. A dental light polymerization device comprising:
an intra-oral tip portion and a handle portion;
a polymerization light source and a camera, wherein the camera has an image input arranged within the tip portion and the polymerization light source comprises a plurality of polymerization light outputs arranged around the image input; and
a light refraction panel in which a plurality of lenses are interspersed, wherein the light refraction panel is arranged with the lenses being each positioned in front of a corresponding polymerization light output of the plurality of polymerization light outputs.

2. The dental light polymerization device of claim 1, wherein the polymerization light outputs are arranged within the tip portion.

3. The dental light polymerization device of claim 1, wherein the lenses are freeform lenses.

4. The dental light polymerization device of claim 1, wherein each lens is based on the same freeform.

5. The dental light polymerization device of claim 1, wherein the lenses have a first optical characteristic, and wherein the light refraction panel comprises a plurality of intermediate portions being arranged between the lenses and having a second optical characteristic that differs from the first optical characteristic.

6. The dental light polymerization device of claim 5, comprising an illumination light source that comprises a plurality of illumination light outputs arranged around the image input.

7. The dental light polymerization device of claim 6, wherein the light refraction panel is arranged with the intermediate portions each positioned in front of a corresponding illumination light output of the plurality of illumination light outputs.

8. The dental light polymerization device of claim 5, wherein the intermediate portions and the lenses are monolithically formed.

9. The dental light polymerization device of claim 5, wherein the first optical characteristic comprises a focusing characteristic that converts a non-parallel light beam from the respective polymerization light output into a substantially parallel light beam.

10. The dental light polymerization device of claim 9, wherein the lenses are oriented such that the light beams exiting the light refraction panel are generally parallel.

11. The dental light polymerization device of claim 5, wherein the intermediate portions are part of a light transmissive plane-parallel portion of the light refraction panel.

12. The dental light polymerization device of claim 5, wherein the second optical characteristic comprises a light dispersion characteristic.

13. The dental light polymerization device of claim 5, wherein each polymerization light output is provided by a blue LED, wherein each illumination light output is provided by a white LED, and wherein the blue and white LEDs and camera are arranged within the tip portion.

14. The dental light polymerization device of claim 13, further comprising a first printed circuit board that carries the white and blue LEDs and the camera, wherein the first printed circuit board is arranged within the tip portion.

15. The dental light polymerization device of claim 1, wherein the light refraction panel has an opening in front of the image input.
